(19) United States
(12) Patent Application Publication
(10) Pub. No.: US 2026/0315277 A1
(43) Pub. Date: Oct. 15, 2026

(54) OPTICAL COHERENCE TOMOGRAPHY (OCT) GUIDANCE USING POLARIZATION DIVERSITY FOR OPHTHALMIC SURGERY

(71) Applicant: Alcon Inc., Fribourg (CH)

(72) Inventors: Corey Stewart, Laguna Niguel, CA (US); Keith Watanabe, Irvine, CA (US); Hongwu Ren, Irvine, CA (US); Alireza Mirsepassi, Irvine, CA (US)

(21) Appl. No.: 19/567,706

(22) Filed: Mar. 16, 2026

Related U.S. Application Data
(60) Provisional application No. 63/789,065, filed on Apr. 15, 2025.

(51) Int. Cl. A61F 9/008

(52) U.S. Cl. CPC A61F 9/008; A61F 2009/00851; A61F 2009/00863; A61F 2009/00874; A61F 2009/00891

(57) ABSTRACT

In certain embodiments, a surgical laser system includes a laser probe and an OCT subsystem. The laser probe includes an optical fiber and an optical tip that includes an optical element that emits an OCT light beam toward tissue and receives reflected light from the tissue to form a sample light beam having line elliptical polarization with two orthogonal polarization states. The OCT subsystem includes an OCT light source that generates the OCT light beam, OCT optical components, and OCT data acquisition components. The OCT optical components transmit the OCT light beam to the optical fiber, generates a reference light beam having elliptical polarization with two orthogonal polarization states, receives the sample light beam from the optical fiber, and generates interference signals based on the polarization states of the reference and sample light beams. The OCT data acquisition components generate image data based on the interference signals.

### INTRODUCTION

The present disclosure relates to surgical systems. More particularly, the present disclosure relates to surgical systems and methods that use Optical Coherence Tomography (OCT) guidance for ophthalmic surgery.

Laser light may be used to treat patients in a wide variety of surgical procedures. For example, a surgical system may use laser light to treat a patient's eye during an ophthalmic surgical procedure, such as glaucoma surgery, vitreoretinal surgery, etc. During glaucoma surgery, laser light may be used to create openings in the tissue in the anterior chamber of the patient's eye. During vitreoretinal surgery, laser light may be used to cut and remove the vitreous and other tissues in the posterior chamber of the patient's eye. Laser light may also be used in other types of ophthalmic surgical procedures, such as cataract surgery, etc.

### SUMMARY

In certain embodiments, a surgical laser system comprises a laser probe and an OCT subsystem. The laser probe comprises an optical fiber and an optical tip. The optical fiber is configured to convey an OCT light beam, and a sample light beam having elliptical polarization comprising two orthogonal polarization states. The optical tip comprises a distal portion of the optical fiber, and at least one optical element configured to emit a first portion of the OCT light beam toward tissue and receive reflected light from the tissue to form the sample light beam. The OCT subsystem is coupled to the laser probe, and comprises an OCT light source, OCT optical components coupled to the OCT light source, and OCT data acquisition components coupled to the OCT optical components. The OCT light source is configured to generate the OCT light beam. The OCT optical components are configured to transmit the OCT light beam from the OCT light source to the optical fiber in the laser probe, generate a reference light beam, based on the OCT light beam, that has elliptical polarization states comprising two orthogonal polarization states, receive the sample light beam from the optical fiber in the laser probe, and generate interference signals based on the polarization states of the reference light beam and the polarization states of the sample light beam. The OCT data acquisition components are configured to generate image data based on the interference signals.

### BRIEF DESCRIPTION OF THE DRAWINGS

FIG. 1A illustrates an example surgical laser system for performing an ophthalmic surgical procedure, in accordance with certain embodiments of the present disclosure.

FIG. 1B illustrates another example surgical laser system for performing an ophthalmic surgical procedure, in accordance with certain embodiments of the present disclosure.

FIG. 2 depicts an example laser probe for a surgical laser system, in accordance with certain embodiments of the present disclosure.

FIG. 3 depicts an example OCT subsystem for a surgical laser system, in accordance with certain embodiments of the present disclosure.

FIG. 4 depicts another example OCT subsystem for a surgical laser system, in accordance with certain embodiments of the present disclosure.

FIG. 5 depicts a flow diagram describing certain functionality of the surgical laser system, in accordance with certain embodiments of the present disclosure.

### DETAILED DESCRIPTION

Embodiments of the present disclosure will now be described with reference to the figures, in which like reference numerals refer to like parts throughout.

A surgical system that uses lasers for treatment is also known as a surgical laser system. Generally, a surgical laser system includes a laser probe and a surgical console. The laser probe includes a handpiece with an optical tip (or head), an optical cable that includes one or more optical fibers, and an optical connector that is attached to, and detached from, an optical port of the surgical console. The optical cable may also include one or more electrical wires, and the optical connector and the optical port may also include one or more electrical connectors. The surgical console houses various subsystems, such as a laser subsystem, an illumination subsystem, a visualization subsystem, etc.

The laser subsystem includes a treatment light source that generates a treatment light beam, such as an infrared (IR) pulsed laser that generates an IR light beam, etc. The treatment light source is optically coupled to the laser probe via the optical port, and the surgeon guides the optical tip of the laser probe handpiece to direct the treatment light beam to the ophthalmic (ocular) tissue. The illumination subsystem includes an illumination light source that generates an illumination light beam, such as one or more continuous wave lasers, etc. The illumination light beam may include one or more colors in the visible spectrum, such as red, blue, green, white, etc., and may also be known as an aiming light beam. The illumination light source is optically coupled to the laser probe via the optical port, and the optical tip of the laser probe handpiece directs the illumination light beam to the ophthalmic tissue. The visualization subsystem may include a camera, a camera interface, and a display. The camera is located within the optical tip of the laser probe handpiece, while the camera interface is located within the surgical console. The camera interface is electrically coupled to the camera via the optical port. The camera provides images of the ophthalmic tissue that is illuminated by the illumination light beam.

In addition to the visualization subsystem, the surgical laser system may include an OCT subsystem to help guide the surgeon during ophthalmic surgery. Generally, the OCT subsystem may provide one dimensional (1D) images, two dimensional (2D) images, and/or three dimensional (3D) images of the ophthalmic tissue using interferometry. The OCT subsystem may generate a 1D image of the ophthalmic tissue by performing a single A scan at a specific location, which provides a depth profile of the ophthalmic tissue at that location. The OCT subsystem may generate a 2D image by performing multiple A scans along a lateral direction (known as a B scan), which provides a cross-sectional view of the ophthalmic tissue along the lateral direction. The OCT subsystem may generate a 3D image by performing multiple B scans across a transverse direction of an area, which provides a volumetric view of the ophthalmic tissue.

The OCT subsystem includes an OCT light source and an OCT detector that are optically coupled to the laser probe via the optical port. The OCT light source generates a coherent light beam, and the optical tip of the laser probe handpiece directs the coherent light beam toward the ophthalmic tissue. The ophthalmic tissue reflects a portion of the coherent light beam back toward the optical tip as a scattered light beam, which is received and conveyed to the OCT detector. The OCT detector measures the interference signal between the reference arm coherent light beam and the sample arm reflected light beam, which is directly related to the depth reflectivity of the ophthalmic tissue. Images of the ophthalmic tissue are generated based on the OCT detector measurements to assist the surgeon in guiding the treatment light beam.

Generally, an OCT subsystem includes a double-path interferometer that generates the interference signal, such as a Michelson interferometer, etc. The double-path interferometer splits the coherent light beam generated by the OCT light source into two separate paths or arms, such as a sample arm and a reference arm.

The sample arm includes an optical path that uses fiber optic light transmission. The length of the sample arm optical path is fixed and extends from the optical tip of the laser probe handpiece through the handpiece, the optical cable, the optical connector, and the optical port, and then through a length of optical fiber that optically couples the optical port to the OCT detector. The sample arm includes a focusing lens that is located at the optical tip of the laser probe handpiece. The coherent light beam from the OCT light source is coupled into the sample arm optical path, and the focusing lens directs the coherent light beam from the OCT light source to the ophthalmic tissue. The focusing lens also receives the light beam that is reflected from the ophthalmic tissue, which is formed into a sample light beam that is conveyed to the OCT detector along the sample arm optical path.

The reference arm includes an optical path that uses free-space light transmission, fiber optic light transmission, or a combination of free-space and fiber optic light transmission. The length of the reference arm optical path is adjustable and extends from the OCT light source to the OCT detector. The reference arm may include an adjustable mirror that reflects the coherent light beam from the OCT light source to the OCT detector as a reference light beam. The optical path length of the reference arm may be increased or decreased by moving the adjustable mirror or corner cube reflector (among others) along a portion of the reference arm optical path to match the optical path length of the sample arm. Alternatively, the reference arm may include a variable delay line (VDL) instead of an adjustable reflector. The VDL can be configured as either reflective type which uses the same port for input and output or transmissive type which uses separate ports for input and output. When the reference arm optical path length and the sample arm optical path length are matched and the polarization states of the reference arm and the sample arm are aligned, then a good interference signal may be generated.

The sample light beam is combined with the reference light beam, and the OCT detector measures the interference signal between the sample light beam and the reference light beam, from which images of the ophthalmic tissue are generated.

When the surgeon guides the laser probe during ophthalmic surgery, this motion bends and twists the optical fiber in the optical cable that conveys the sample light beam from the optical tip of the laser probe handpiece to the optical port of the surgical console. Unfortunately, bending and twisting the optical fiber in the sample arm introduces a change in birefringence of the optical fiber in the optical cable, which changes the polarization states of the sample light beam in the sample arm. The difference in the polarization states between the reference light beam in the reference arm and the sample light beam in the sample arm will randomly change the amplitude of the optical signal measured by the OCT detector. This amplitude instability in the optical signal measured by the OCT detector degrades the quality of the images that are generated based on the OCT measurements (such as image clarity, fidelity, signal-to-noise ratio (SNR), etc.). Poor quality OCT-based images impair the guidance of the laser probe during ophthalmic surgery.

The reference light beam and the sample light beam may be elliptical polarized, and each may be described by two orthogonal polarization components or states, such as a horizontal polarization state and a vertical polarization state, or a P-polarization state (or polarization state P) and an S-polarization state (or polarization state S). The horizontal and vertical polarization states are defined with respect to a reference set of orthogonal axes (such as an x-axis and a y-axis), while the polarization states P and S are defined with respect to a reference incident plane. The polarization state P refers to the polarization component in the plane, and the polarization state S refers to the polarization component perpendicular to the plane.

A polarization state controller may be used in the reference arm (or the sample arm) to match the polarization states of the reference light beam and the sample light beam. For example, the polarization state controller may include two or three loops of optical fiber that are wrapped around a spool and rotated or stretched to change the polarization states of the light beam. However, the changes in the polarization states of the sample light beam caused by the movement of the laser probe by the surgeon exceed the rate at which the polarization state controller can change the polarization states by moving the loops of optical fiber. In other words, the polarization state controller cannot move the loops of optical fiber fast enough to stabilize the OCT signal due to the bending and twisting of the optical fiber in the laser probe.

Embodiments of the present disclosure advantageously provide an OCT system that uses a polarization diversity detection architecture that reduces or eliminates the effect from polarization differences caused by bending and twisting the optical fiber in the sample arm, which reduces or eliminates the amplitude instability of the optical signal measured by the OCT detector thereby improving the quality of the images that are generated based on the OCT measurements.

While described in the context of an ophthalmic surgical procedure, the systems and methods described herein may be used for any suitable medical procedure that uses laser light for treatment purposes, such as tumor removal, wound and scar treatments, dermatology procedures, etc.

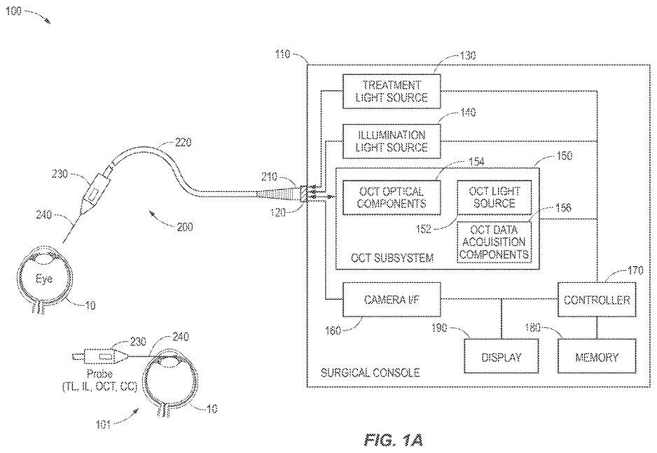

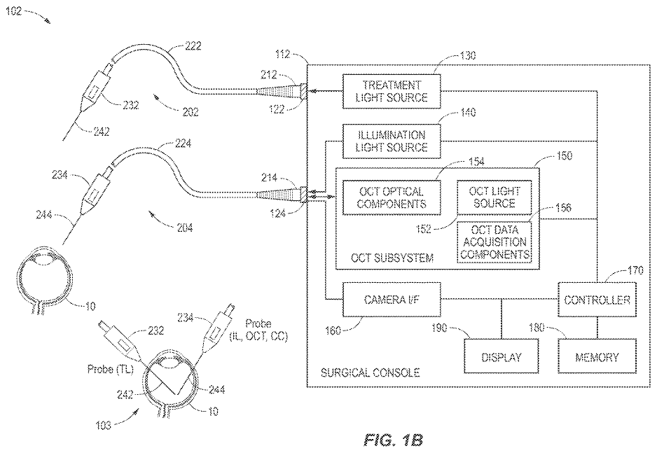

FIG. 1A illustrates an example surgical laser system 100 for performing a laser-assisted ophthalmic surgical procedure on a patient's eye 10, in accordance with certain embodiments of the present disclosure.

In certain embodiments, the surgical laser system 100 includes, among other things, a surgical console 110 and a laser probe 200. Generally, the surgical laser system 100 may be used in a variety of ophthalmic surgical procedures, such as glaucoma surgery, vitreoretinal surgery, cataract surgery, etc. In certain embodiments, the surgical laser system 100 may be configured to support a particular ophthalmic surgical procedure.

The surgical console 110 may include, among other things, an electro-optical (EO) port 120, a treatment light source 130, an illumination light source 140, an OCT subsystem 150, a camera interface (I/F) 160, a controller 170, a memory 180, and a display 190. The surgical console 110 may also include additional tools and subsystems to facilitate the performance of the ophthalmic surgical procedure. One example of a surgical console configured for performing anterior and posterior segment ophthalmic surgery is the Constellation® System available from Alcon Laboratories, Inc., Fort Worth, Texas.

The EO port 120 is configured to receive and secure the EO connector 210 of the laser probe 200. The EO port 120 is optically coupled to the treatment light source 130, the illumination light source 140, and the OCT subsystem 150 using separate optical fibers, such as a treatment optical fiber, an illumination optical fiber, etc. The EO port 120 is electrically coupled to the camera I/F 160 using electrical wires.

The treatment light source 130 is configured to generate laser light that is formed into a treatment light beam. In certain embodiments, the treatment light source 130 is a pulsed laser that generates a treatment light beam in the IR spectrum (for example, about 750 nm (nanometers) to 3000 nm) in response to control signals provided by the controller 170. The treatment light beam includes short, periodic IR laser pulses, such as 2-5 nanosecond pulses at a 1 kHz (kilohertz) pulse rate. Each IR laser pulse may have a pulse shape that is defined by the intensity of the IR laser pulse over the duration of the pulse, such as a “Gaussian” pulse shape.

The illumination light source 140 is configured to generate light that is formed into an illumination light beam. In certain embodiments, the illumination light source 140 includes three continuous wave (CW) lasers that generate laser light beams in the red, green, and blue spectrums in response to control signals provided by the controller 170. The red, green, and blue laser light beams are optically combined to form a white (or pseudo-white) illumination light beam in the visible spectrum (for example, about 380 nm to 750 nm). Other visible spectrum (color) combinations may also be used. In other embodiments, the illumination light source 140 includes three light emitting diodes (LEDs) that generate light beams in the red, green, and blue spectrums in response to control signals provided by the controller 170. The red, green, and blue light beams are optically combined to form a white (or pseudo-white) illumination light beam in the visible spectrum. In some embodiments, the illumination light source 140 includes a single CW laser or LED that generates a monochromatic light beam, such as a light beam in the red spectrum, the green spectrum, the blue spectrum, etc.

The OCT subsystem 150 may include an OCT light source 152, OCT optical components 154, and OCT data acquisition components 330.

The OCT subsystem 150 uses a polarization diversity detection architecture with separate optical paths for the reference and sample arms and processes the interference signals in the Fourier or frequency domain (FD).

The OCT light source 152 is optically coupled to the OCT optical components 154 using an optical fiber and electrically coupled to the OCT data acquisition components 156 using electrical wires or cables.

In certain embodiments, the OCT subsystem 150 is a swept source OCT (SS-OCT) subsystem, and the OCT light source 152 is a swept-source laser that generates a coherent light beam that is rapidly swept (or varied) over a broad range of wavelengths over a short period of time. The light reflected from the ophthalmic tissue at each wavelength generates an interference fringe when combined with a portion of the coherent light beam, and the interference fringes are detected and processed to generate an image (such as a 1D image, a 2D image, or a 3D image).

In other embodiments, the OCT subsystem 150 is a spectral domain OCT (SD-OCT) subsystem, and the OCT light source 152 is a broadband light source (such as a superluminescent diode) that generates a broadband light beam. The broad spectrum of wavelengths produces simultaneous reflection of light from different depths of the ophthalmic tissues. The light reflected from the ophthalmic tissue generates interference fringes when combined with a portion of the broadband light beam, which are detected and processed to generate an image.

The OCT optical components 154 include optical and electro-optical components that guide and adjust the outgoing and incoming light beams and generate and detect interference signals. The OCT optical components 154 are optically coupled to the EO port 120 using an optical fiber and electrically coupled to the OCT data acquisition components 156 using electrical wires or cables.

Generally, the OCT light source 152 generates an OCT light beam that is provided to the OCT optical components 154, such as a coherent light beam from a swept-source laser or a broadband light beam from a broadband light source. The OCT optical components 154 send the OCT light beam to the laser probe 200 and generate a reference light beam based on the OCT light beam. The reference light beam has elliptical polarization with two orthogonal polarization states. The laser probe 200 emits the OCT light beam from an optical tip 240 toward the ophthalmic tissue, receives the light reflected by the ophthalmic tissue, and sends the light reflected by the ophthalmic tissue from the optical tip 240 of the laser probe 200 to the OCT optical components 154 as a sample light beam. The sample light beam has elliptical polarization with two orthogonal polarization states. The OCT optical components 154 also generate interference signals based on the orthogonal polarization states of the reference and sample light beams and send the interference signals to the OCT data acquisition components 156.

Advantageously, the OCT optical components 154 use a polarization diversity detection architecture with separate optical paths for the reference and sample arms that reduces or eliminates the effect from polarization differences caused by bending and twisting the optical fiber in the laser probe 200. The reference light beam and the sample light beam are provided to a detection arm, which generates two sets of spectral fringes, one set of spectral fringes for each orthogonal polarization state.

The OCT data acquisition components 156 may include a processor, memory, and signal conditioning and processing circuitry, such as an analog-to-digital (A/D) converter, a video frame grabber card, etc. Generally, the OCT data acquisition components 156 accept the trigger and clock control signals from the OCT light source 152, acquire and process the interference signals, generate the images, and send the images to the controller 170 for further processing.

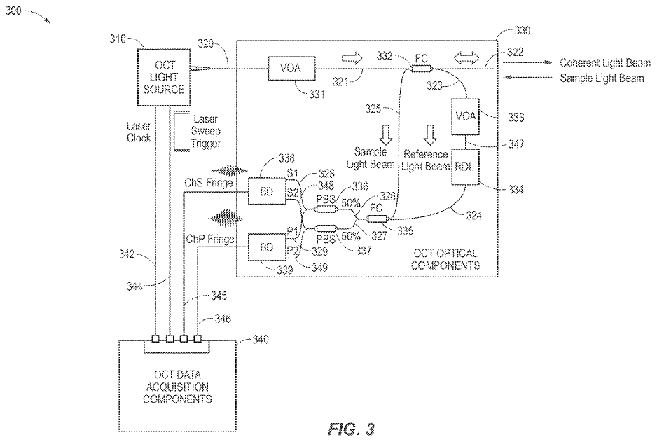

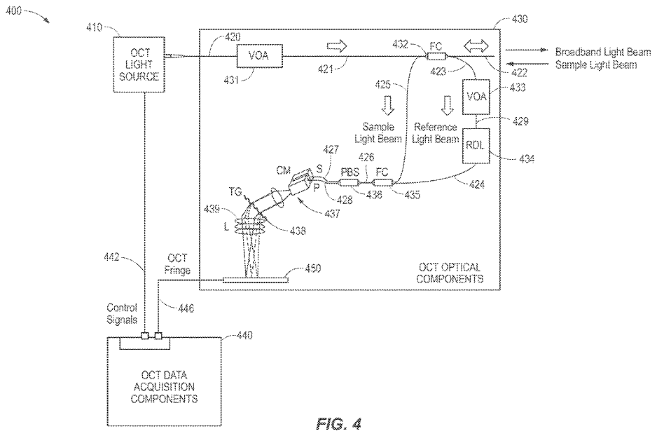

An example of an SS-OCT subsystem using a polarization diversity detection architecture is described in more detail below with respect to FIG. 3, and an example of an SD-OCT subsystem using a polarization diversity detection architecture is described in more detail below with respect to FIG. 4.

The controller 170 is coupled to the treatment light source 130, the illumination light source 140, the OCT subsystem 150, the camera I/F 160, the memory 180, and the display 190. The controller 170 may be a microcontroller, a microprocessor, a programmable logic controller (PLC), a field programmable gate array (FPGA), an application specific integrated circuit (ASIC), etc.

Generally, the controller 170 is configured to control the operation of the treatment light source 130 through various settings (such as pulse width or duration, pulse frequency or period, wavelength, power or intensity, spot size, etc.), and to generate control signals for the treatment light source 130. The controller 170 is also configured to control the operation of the illumination light source 140 through various settings (such as power or intensity, wavelength, etc.), and to generate control signals for the illumination light source 140. The controller 170 is also configured to control the operation of the OCT subsystem 150, to receive and process image data from the OCT subsystem 150, and to present the image data on the display 190. The controller 170 is also configured to control to receive and process image data from the camera I/F 160 and present the image data on the display 190.

The laser probe 200 includes, among other things, the EO connector 210, an EO cable 220, and a handpiece 230 including an optical tip 240. The distal end of the EO cable 220 may be directly attached to the handpiece 230; alternatively, the distal end of the EO cable 220 may be removably coupled to the handpiece 230 using an EO connector. The proximal end of the EO cable 220 includes the EO connector 210, which may be attached to, and detached from, the EO port 120. Several optical fibers and electrical wires within the EO cable 220 and the handpiece 230 couple the EO connector 210 to the optical tip 240.

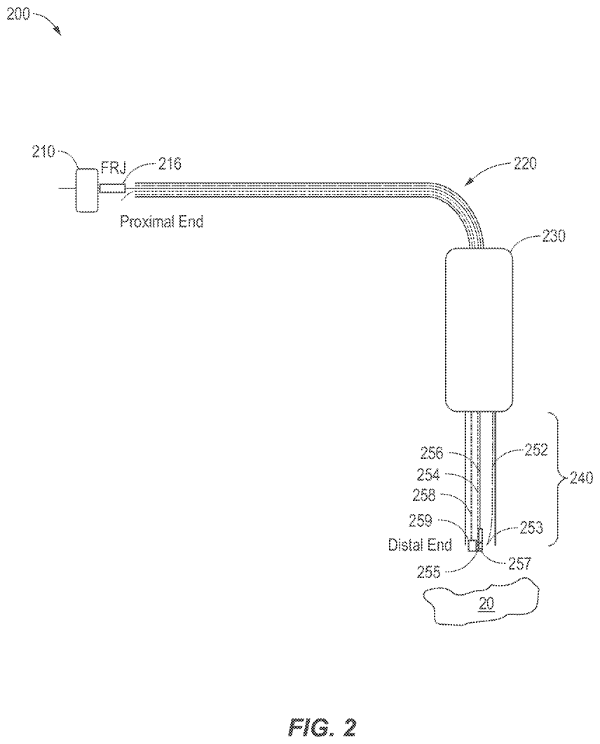

FIG. 2 depicts an example laser probe 200 for a surgical laser system 100, in accordance with certain embodiments of the present disclosure.

In certain embodiments, the EO cable 220 and the handpiece 230 include optical fibers 252, 254, 256. In certain embodiments, the optical fibers may include single-core optical fibers (SCFs), multi-core optical fibers (MCFs), such as GeO2 with tapped sapphire cable, a combination of single-core and multi-core fibers, etc. Other fiber optical materials may also be used, such as Ti: Sapphire, Y3Al5O12 (YAG), Ho:YAG, Yb:YAG, Nd:YAG, Er:YAG, Ce:YAG, Cr:YAG, or ZrF4-BaF2-LaF3-AlF3-NaF (ZBLAN), etc.

The optical fiber 252 conveys the treatment light beam from the EO connector 210 to the optical tip 240 and may have an optical element 253 (such as a focusing lens, a lensed tip, etc.) attached to, or formed in, the distal end.

The optical fiber 254 conveys the illumination light beam from the EO connector 210 to the optical tip 240 and may have an optical element 255 (such as focusing lens, a lensed tip, etc.) attached to, or formed in, the distal end.

The optical fiber 256 conveys the OCT light beam from the EO connector 210 to the optical tip 240 and conveys the sample light beam from the optical tip 240 to the EO connector 210. The optical fiber 256 may have at least one optical element 257 attached to, or formed in, the distal end.

For example, the optical element 257 may include a polished angled tip that is coated with an anti-reflection coating. The angled polished tip emits the OCT light beam towards the ophthalmic tissue 20 and receives the reflected light from the ophthalmic tissue 20, which is formed into the sample light beam. In another example, the optical element 257 may include a focusing lens (such as a gradient-index or GRIN lens) that has an anti-reflective coating. The focusing lens may be attached to the end of the optical fiber 256 using an optical adhesive that has a refractive index that matches the refractive index of the optical fiber 256.

In some embodiments, a 2×1 fiber coupler may combine the treatment light beam transmitted in one optical fiber and the illumination light beam transmitted in another optical fiber into a single optical fiber that is coupled to the EO port 120. The optical fiber 254 then conveys the treatment light beam and the illumination light beam from the EO connector 210 to the optical tip 240, and the optical fiber 252 is not needed.

In other embodiments, a 2×1 fiber coupler combines the illumination light beam transmitted in one optical fiber and the OCT light beam transmitted in another optical fiber into a single optical fiber that is coupled to the EO port 120. The optical fiber 256 then conveys the illumination light beam and the OCT light beam from the EO connector 210 to the optical tip 240, and the optical fiber 254 is not needed.

In certain embodiments, the OCT subsystem 150 may generate a 1D image of the ophthalmic tissue 20 using an A scan. The 1D image may be used to determine the distance from the optical tip 240 to the ophthalmic tissue 20. The A scan does not require displacement of the OCT light beam along lateral or transverse directions.

In other embodiments, the OCT subsystem 150 may generate a 2D image of the ophthalmic tissue 20 using a B scan, which performs multiple A scans along a lateral direction. The B scan requires incremental displacement of the OCT light beam along the lateral direction for each A scan.

In one example, a fiber rotary joint (FRJ) 216 may be coupled to the EO connector 210 and to the proximal end of the optical fiber 256. A motor drive coupled to the FRJ 216 rotates the optical fiber 256 and the optical element 257 between a positive displacement angle (such as +45°) and a negative displacement angle (such as −45°) in a lateral direction to perform the A scans that comprise the B scan. The motor drive may be coupled to, and controlled by, the OCT data acquisition components 156. In another example, the optical element 257 may also include a deflection element, such as an articulated PZT (Lead Zirconate Titanate) or MEMS (Micro-Electro-Mechanical Systems) mirror, an articulated prism, etc., that moves the OCT light beam between a positive displacement angle (such as +45°) and a negative displacement angle (such as −45°) in a lateral direction to perform the A scans that comprise the B scan. The deflection element may be coupled to, and controlled by, the OCT data acquisition components 156. Other displacement angles may also be used, such as ±30°, ±15°, etc.

In some embodiments, the OCT subsystem 150 may generate a 3D image of the ophthalmic tissue 20 by displacing the deflection element along a transverse direction to perform the B scans that comprise the volumetric scan.

The EO cable 220 and the handpiece 230 also include a number of electrical wires 258. The electrical wires 258 may include, among other things, power wires to convey positive and negative DC voltages from the EO connector 210 to a camera 259 located in the optical tip 240, one or more signal wires to convey video signals from the camera 259 to the EO connector 210, etc. The illumination light beam illuminates the ophthalmic tissue 20 for the camera 259. In some embodiments, the camera may be located in the camera I/F 160, and the EO cable 220 and the handpiece 230 may include an optical fiber bundle that conveys an optical image from the optical tip 240 to the EO connector 210. The EO port 120 may be optically coupled to the camera I/F 160 using an optical fiber bundle.

Referring back to FIG. 1A, the surgeon may activate and deactivate the treatment light source 130 using a mechanical or electrical control, such as a foot pedal, a switch on the handpiece 230, etc. The mechanical or electrical control may be coupled to the controller 170 or directly to the treatment light source 130. In one example, an electrical cable may couple the foot pedal to the controller 170. In another example, one or more electrical wires located within the EO cable 220 and handpiece 230 may couple the switch to the EO connector 210.

The laser probe 200 may be used during glaucoma surgery 101 in the anterior chamber of the eye 10 to treat different areas in the trabecular meshwork spatially adjacent to the Schlemm's canal. During glaucoma surgery 101, the optical tip 240 is inserted into the anterior chamber of the eye 10, and the surgeon directs the treatment light beam to different areas in the trabecular meshwork spatially adjacent to the Schlemm's canal, guided by the images generated by OCT subsystem 150 and the images generated by the camera in the optical tip 240 that are presented on the display 190.

FIG. 1B illustrates another example surgical laser system 102 for performing a laser-assisted ophthalmic surgical procedure on a patient's eye 10, in accordance with certain embodiments of the present disclosure.

In certain embodiments, the surgical laser system 102 includes, among other things, a surgical console 112, a treatment laser probe 202, and a visualization laser probe 204. Generally, the components of the laser probe 200 described above are divided between the treatment laser probe 202 and the visualization laser probe 204, and the EO port 120 is replaced by an optical port 122 and an EO port 124. In certain embodiments, the treatment laser probe 202 includes the components related to the treatment light source 130, while the visualization laser probe 204 includes the components related to the illumination light source 140, the OCT subsystem 150, and the camera and camera I/F 160. The surgical laser system 102 may also be used in a variety of ophthalmic surgical procedures, such as glaucoma surgery, vitreoretinal surgery, cataract surgery, etc.

The surgical console 112 may include, among other things, the treatment light source 130, the illumination light source 140, the OCT subsystem 150, the camera I/F 160, the controller 170, the memory 180, and the display 190, as described above with respect to the surgical console 110, as well as the optical port 122 and the EO port 124. The surgical console 112 may also include additional tools and subsystems to facilitate the performance of the ophthalmic surgical procedure.

The optical port 122 is configured to receive and secure the optical connector 212 of the treatment laser probe 202. The optical port 122 is optically coupled to the treatment light source 130 using an optical fiber.

The EO port 124 is configured to receive and secure the EO connector 214 of the visualization laser probe 204. The EO port 124 is optically coupled to the illumination light source 140 and the OCT subsystem 150 using separate optical fibers and is electrically coupled to the camera I/F 160 using electrical wires.

The treatment laser probe 202 includes, among other things, the optical connector 212, an optical cable 222, and a handpiece 232 including an optical tip 242. The distal end of the optical cable 222 may be directly attached to the handpiece 232; alternatively, the distal end of the optical cable 222 may be removably coupled to the handpiece 232 using an optical connector. The proximal end of the optical cable 222 includes the optical connector 212, which may be attached to, and detached from, the optical port 122.

An optical fiber within the optical cable 222 and the handpiece 232 couples the optical connector 212 to the optical tip 242 and conveys the treatment light beam from the optical connector 212 to the optical tip 242, as discussed above with respect to the optical fiber 252. The optical fiber may have an optical element (such as a focusing lens, a lensed tip, etc.) attached to, or formed in, the distal end, as described above with respect to the optical element 253.

The visualization laser probe 204 includes, among other things, the EO connector 214, an EO cable 224, and a handpiece 234 including an optical tip 244. The distal end of the EO cable 224 may be directly attached to the handpiece 234; alternatively, the distal end of the EO cable 224 may be removably coupled to the handpiece 234 using an EO connector. The proximal end of the EO cable 224 includes the EO connector 214, which may be attached to, and detached from, the EO port 124.

Several optical fibers and electrical wires within the EO cable 224 and handpiece 234 couple the EO connector 214 to the optical tip 244.

One optical fiber conveys the illumination light beam from the EO connector 214 to the optical tip 244, as discussed above with respect to the optical fiber 254. This optical fiber may have an optical element (such as a focusing lens, a lensed tip, etc.) attached to, or formed in, the distal end, as described above with respect to the optical element 255.

Another optical fiber conveys the OCT light beam from the EO connector 214 to the optical tip 244 and conveys the sample light beam from the optical tip 244 to the EO connector 214. This optical fiber may have one or more optical elements attached to, or formed in, the distal end, as described above with respect to the optical element 257.

The EO cable 224 and the handpiece 234 also include a number of electrical wires, as described above with respect to electrical wires 258. The electrical wires may include, among other things, power wires to convey positive and negative DC voltages from the EO connector 214 to a camera located in the optical tip 244, one or more signal wires to convey video signals from the camera to the EO connector 214, etc. The illumination light beam illuminates the ophthalmic tissue for the camera. In some embodiments, the camera may be located in the camera I/F 160, and the EO cable 224 and the handpiece 234 may include an optical fiber bundle that conveys an optical image from the optical tip 244 to the EO connector 214. The EO port 124 may be optically coupled to the camera I/F 160 using an optical fiber bundle

The treatment laser probe 202 and the visualization laser probe 204 may be used during vitreoretinal surgery 103 in the posterior chamber of the eye 10. During vitreoretinal surgery 103, the optical tip 242 of the treatment laser probe 202 and the optical tip 244 of the visualization laser probe 204 are inserted into the posterior chamber of the eye 10, and the surgeon independently directs the treatment light beam emitted by the optical tip 242 and the illumination light beam emitted by the optical tip 244 and toward the ophthalmic tissue, guided by the images generated by OCT subsystem 150 and the images generated by the camera in the optical tip 244 that are presented on the display 190. The laser probe 200 of the surgical laser system 100 (FIG. 1A) may also be used for vitreoretinal surgery 103 in the posterior chamber of the eye 10.

In certain embodiments, the treatment light source 130, the optical port 122, and the treatment laser probe 202 may be replaced by an ultrasonic source, an electrical port, and an ultrasonic probe, respectively. The electrical port may be configured to receive and secure the electrical connector of the ultrasonic probe and is electrically coupled to the ultrasonic source using an electrical cable. The ultrasonic probe may include the electrical connector, an electrical cable, and a handpiece with an ultrasonic tip. The ultrasonic probe and the visualization laser probe 204 may be cooperatively used during vitreoretinal surgery 103 in the posterior chamber of the eye 10.

As discussed above, in certain embodiments, the OCT subsystem 150 may be an SS-OCT subsystem.

FIG. 3 depicts an example SS-OCT subsystem 300 for the surgical laser systems 100, 102, in accordance with certain embodiments of the present disclosure.

In certain embodiments, the SS-OCT subsystem 300 includes, among other things, an OCT light source 310, OCT optical components 330, and OCT data acquisition components 340. The SS-OCT subsystem 300 uses a polarization diversity detection architecture and processes the interference signals in the Fourier or frequency domain (FD).

The OCT light source 310 is a swept-source laser that generates a coherent light beam that is rapidly swept (or varied) over a broad range of wavelengths over a short period of time, as discussed above.

Generally, the OCT optical components 330 include optical and electro-optical components that include, among other things, a variable optical attenuator (VOA) 331, a fiber coupler (FC) 332, a VOA 333, a reference delay line (RDL) 334, a fiber coupler 335, a polarization beam splitter (PBS) 336, a PBS 337, a balance detector (BD) 338, and a balance detector 339, which are optically coupled using optical fibers.

The OCT light source 310 is coupled to the input port of the VOA 331 via the optical fiber 320. The VOA 331 is a passive optical device that reduces the optical power of the coherent light beam to prevent signal saturation during acquisition, and for optimization of the system sensitivity. The amount of attenuation provided by the VOA 331 may be configurable. The output port of the VOA 331 is coupled to an input port of the fiber coupler 332 via optical fiber 321.

The fiber coupler 332 is a “2×2” fiber coupler with two input ports and two output ports. A first input port receives the coherent light beam from the VOA 331 and passes the coherent light beam to a first output port and a second output port. The first output port is coupled to the EO port 120 of the surgical console 110 (or the EO port 124 of the surgical console 112) via the optical fiber 322. When the EO connector 210 of the laser probe 200 is attached to the EO port 120, the optical fiber 322 is optically coupled to the optical fiber 256 of the laser probe 200. The second output port is coupled to the VOA 333 via the optical fiber 323.

In addition to transmitting the coherent light beam to the EO port 120, the first output port of the fiber coupler 332 also receives the sample light beam from the EO port 120. The fiber coupler 332 passes the sample light beam to the second input port (which functions as an output port), which is coupled to a first input port of the fiber coupler 335 via the optical fiber 325. In other words, the fiber coupler 332 combines the coherent light beam travelling in one direction with the sample light beam travelling in the other direction.

The optical path for the sample arm of the SS-OCT subsystem 300 includes the optical fiber 256, the optical fiber 322, the fiber coupler 332, and the optical fiber 325. In certain embodiments, the sample arm begins at the optical element 257 and ends at the fiber coupler 335.

The optical path for the reference arm of the SS-OCT subsystem 300 begins at the fiber coupler 332, includes the optical fiber 323, the VOA 333, the RDL 334, and the optical fiber 324, and ends at the fiber coupler 335. The optical fiber 323 is coupled to the input port of the VOA 333. The VOA 333 is also a passive optical device that reduces the optical power of the coherent light beam to prevent signal saturation during acquisition and for optimization of the system sensitivity. The amount of attenuation provided by the VOA 333 may be configurable. The output port of the VOA 333 is coupled to the input port of the RDL 334, either directly or via an optical fiber 347. The RDL 334 provides a precise, adjustable time delay to the reference light beam to align the reference arm for accurate depth imaging.

The optical path for the detection arm begins at the fiber coupler 335, includes the optical fibers 326, 327, the PBSs 336, 337, and the optical fibers 328, 329, 348, 349, and ends at the balanced detectors 338, 339.

The fiber coupler 335 is a “2×2” (50/50) fiber coupler that combines the reference light beam and the sample light beam and divides the combined light beams into two equal light beams. The fiber coupler 335 includes two input ports and two output ports. The first input port receives the sample light beam from the optical fiber 325, and the second input port receives the reference light beam from the optical fiber 324. The fiber coupler 335 divides the reference and sample light beams into two equal light beams, which are provided to the first output port and the second output port, respectively. The first output port is coupled to the input port of the PBS 336 via optical cable 326, and the second output port is coupled to the input port of the PBS 337 via optical cable 327.

The PBS 336 divides the first light beam received by the input port into an S₁ polarization channel light beam and a P₁ polarization channel light beam. The S₁ polarization channel light beam is provided to the first output port, which is coupled to the first input port of the balanced detector 338 via optical fiber 328. The P₁ polarization channel light beam is provided to the second output port, which is coupled to the first input port of the balanced detector 339 via optical cable 329. In certain embodiments, the optical fiber or optical cables after PBS 336 may be polarization maintaining fibers.

The PBS 337 divides the second light beam received by the input port into an S₂ polarization channel light beam and a P₂ polarization channel light beam. The S₂ polarization channel light beam is provided to the first output port, which is coupled to the second input port of the balanced detector 338 via optical cable 348. The P₂ polarization channel light beam is provided to the second output port, which is coupled to the second input port of the balanced detector 339 via optical fiber 349. In certain embodiments, the optical fiber or optical cables after PBS 337 may be polarization maintaining fibers.

In certain embodiments, to optimize the signal to noise ratio in the OCT complex amplitude signals Is or Ip, a polarization controller may be used in the reference arm or the sample arm between fiber coupler 332 and fiber coupler 335 in FIG. 3, and between fiber coupler 432 and fiber coupler 435 in FIG. 4. The polarization controller does not need to move during imaging and is configured to optimize the signal to noise ratio of OCT signal I before start of the imaging.

The balanced detectors 338, 339 include a photodetector coupled to each input port, and signal processing components coupled to the photodetectors. Each photodetector detects the fringe intensity of the respective polarized input light beams and generates an electrical signal (such as a voltage signal) that is proportional to the fringe intensity. The signal processing components electronically subtract the electrical signals from the photodetectors to generate a difference signal that is cleaner and less noisy than the input electrical signals. In one example, the signal processing components may include a differential amplifier circuit, which applies the electrical current signals from the photodetectors to the inverting and non-inverting inputs of a transimpedance amplifier (a type of op-amp) to convert the electrical current signal to voltage signal. The output voltage of the op-amp represents the difference between the electrical signals from the photodetectors.

The balanced detector 338 provides the S polarization channel interference signal to the OCT data acquisition components 340 via the electrical cable 345, while the balanced detector 339 provides the P polarization channel interference signal to the OCT data acquisition components 340 via the electrical cable 346.

The OCT data acquisition components 340 may include a processor, memory, and signal conditioning and processing circuitry, such as an analog-to-digital (A/D) converter, field programmable gate array (FPGA), etc. Generally, the OCT data acquisition components 340 accept the control signals from the OCT light source 310, process the interference signals from the balanced detector 338 and 339, generate images based on the processed interference signals, and send the images to the controller 170 for further processing.

In certain embodiments, the OCT data acquisition components 340 may digitize and then perform dispersion compensation, apodization windowing, and fast Fourier transforms (FFTs) on the S polarization channel interference signal to generate OCT complex amplitude signal Is, and may digitize and then perform the same processing procedures on the P polarization channel interference signal to generate OCT complex amplitude signal Ip. The OCT data acquisition components 340 may combine the Is and Ip OCT signals to generate the OCT signal I, as given by Equation 1, where ||denote the absolute values of complex numbers.

I=\sqrt{|Is|²+|Ip|²} Eq. 1

The OCT data acquisition components 340 may then perform logarithm scale calculation on OCT signal I to generate A scan intensity profiles for the images.

The control signals from the OCT light source 310 may include a laser clock signal conveyed via the electrical cable 342, and a laser sweep trigger (axial scan trigger) signal conveyed via the electrical cable 344. The overall operation of the SS-OCT subsystem 300 is controlled by controller 170, which sends control signals to the OCT data acquisition components 340 over electrical wires or cables.

As discussed above, in certain embodiments, the OCT subsystem 150 may be an SD-OCT subsystem.

FIG. 4 depicts an example SD-OCT subsystem 400 for the surgical laser systems 100, 102, in accordance with certain embodiments of the present disclosure.

In certain embodiments, the SD-OCT subsystem 400 includes, among other things, an OCT light source 410, OCT optical components 430, and OCT data acquisition components 440. The SD-OCT subsystem 400 uses a polarization diversity detection architecture and processes the interference signals in the Fourier or frequency domain (FD).

The OCT light source 410 is a broadband light source (such as a superluminescent diode) that generates a broadband light beam, as described above.

Generally, the OCT optical components 430 include optical and electro-optical components that include, among other things, a VOA 431, a fiber coupler 432, a VOA 433, an RDL 434, a fiber coupler 435, and a PBS 436, which are optically coupled using optical fibers. The OCT optical components 430 also include several free space optical components, such as a collimator (or collimator components) 437, a transmission grating (TG) 438, an imaging lens (L) 439, and an image capture device 450, such as an array of line array charge-coupled devices (CCD) (each having 1024 pixels, 2048 pixels, etc.), a complementary metal-oxide semiconductor (CMOS) camera having n×1024 pixels, n×2048 pixels, where n≥2, etc.

The OCT light source 410 is coupled to the input port of the VOA 431 via the optical fiber 420. The VOA 431 is a passive optical device that reduces the optical power of the broadband light beam to prevent signal saturation during acquisition. The amount of attenuation provided by the VOA 431 may be configurable. The output port of the VOA 431 is coupled to an input port of the fiber coupler 432 via optical fiber 421.

The fiber coupler 432 is a “2×2” fiber coupler with two input ports and two output ports. A first input port receives the coherent light beam from the VOA 431 and passes the coherent light beam to a first output port and a second output port. The first output port is coupled to the EO port 120 of the surgical console 110 (or the EO port 124 of the surgical console 112) via the optical fiber 422. When the EO connector 210 of the laser probe 200 is attached to the EO port 120, the optical fiber 422 is optically coupled to the optical fiber 256 of the laser probe 200. The second output port is coupled to the VOA 433 via the optical fiber 423.

In addition to transmitting the coherent light beam to the EO port 120, the first output port of the fiber coupler 432 also receives the sample light beam from the EO port 120. The fiber coupler 432 passes the sample light beam to the second input port (which functions as an output port), which is coupled to a first input port of the fiber coupler 435 via the optical fiber 425. In other words, the fiber coupler 432 combines the coherent light beam travelling in one direction with the sample light beam travelling in the other direction.

The optical path for the sample arm of the SD-OCT subsystem 400 includes the optical fiber 256, the optical fiber 422, the fiber coupler 432, and the optical fiber 425. In certain embodiments, the sample arm begins at the optical element 257 and ends at the fiber coupler 435.

The optical path for the reference arm of the SD-OCT subsystem 300 begins at the fiber coupler 432, includes the optical fiber 423, the VOA 433, the RDL 434, and the optical fiber 424, and ends at the fiber coupler 435. The optical fiber 423 is coupled to the input port of the VOA 433. The VOA 433 is also a passive optical device that reduces the optical power of the coherent light beam to prevent signal saturation during acquisition and for optimization of the system sensitivity. The amount of attenuation provided by the VOA 433 may be configurable. The output port of the VOA 433 is coupled to the input port of the RDL 434, either directly or via an optical fiber 429. The RDL 434 provides a precise, adjustable time delay to the reference light beam to align the reference arm for accurate depth imaging.

The fiber optic portion of the optical path for the detection arm begins at the fiber coupler 435, includes the optical fiber 426, the PBS 436, and the optical fibers 427, 428, and ends at the inputs to the collimator components 437. The free space portion of the optical path for the detection arm begins at the collimator components 437, includes the transmission grating 438, imaging lens 439, and ends at the imaging capture device 450.

The fiber coupler 435 is a “2×1” fiber coupler that combines the reference light beam and the sample light beam. The fiber coupler 435 includes two input ports and one output port. The first input port receives the reference light beam from the optical fiber 424, and the second input port receives the sample light beam from the optical fiber 425. The output port is coupled to the input port of the PBS 436 via optical cable 426.

The PBS 436 divides the combined light beam received by the input port into an S polarization channel light beam and a P polarization channel light beam. The S polarization channel light beam is provided to the first output port, which is coupled to the first input port of the collimator components 437 via optical cable 427. The P polarization channel light beam is provided to the second output port, which is coupled to the second input port of the collimator components 437 via optical cable 428.

The collimator components 437 have two collimators, and each collimator has an input port, an aperture, and a lens. The S polarization channel light beam is emitted from the aperture of the first collimator as a free space divergent light beam, and the P polarization channel light beam is emitted from the aperture of the second collimator as a free space divergent light beam. Each free space divergent light beam is directed onto a respective lens, which focuses the free space divergent light beam into a free space collimated light beam. The lens is positioned at a particular distance relative to the aperture to provide the proper collimation. Generally, the aperture and the lens are contained within a housing, and each collimator may be adjustable to vary the free space collimated light beam for the best collimation.

The collimators are tilted with respect to each other to separate the optical beams of S and P polarization channel light beams on the two line arrays of the imaging capture device 450 for detecting the two sets of spectral fringes of S and P polarization states after going through the transmission grating 438 and imaging lens 439.

The transmission grating 438 is a type of diffraction grating in which the incident light beam passes through the grating structure (rather than reflecting off of it) and is diffracted into multiple light beams based on wavelength. The transmission grating 438 receives the free space collimated light beams for the S and P polarization channels, each of which is diffracted into a number of separate light beams detected on pixels of each of the two-line arrays of the imaging capture device 450 with pixel height larger than spot diameter of each light beam to suppress the crosstalk between the S and P polarization channels.

The imaging lens 439 includes one or more lenses that focuses the light beams generated by the transmission grating 438 onto the image capture device 450.

The image capture device 450 converts the light beams for each wavelength (or wavelength region) into electrical signals, which are provided to the OCT data acquisition components 440 as interference signals via the electrical cable 446. Since two sets of polarization channel light beams are detected, multiple line array CCD devices, or a CMOS camera with two dimensional sensor, are used since there may need a space between the two line arrays to suppress the crosstalk of two sets of polarization states.

The OCT data acquisition components 440 may include a processor, memory, and signal conditioning and processing circuitry, such as an analog-to-digital (A/D) converter, a video frame grabber card, etc. Generally, the OCT data acquisition components 440 accept the trigger or other control signals from the OCT light source 410, process the interference signals from the image capture device 450, generate images based on the processed interference signals, and send the images to the controller 170 for further processing.

In certain embodiments, the OCT data acquisition components 440 first digitize the interference signals. The OCT data acquisition components 440 may perform dispersion compensation, apodization windowing, and FFTs on the S polarization channel interference signal to generate OCT complex amplitude signal Is. The OCT data acquisition components 440 may perform the same processing procedures on the P polarization channel interference signal to generate OCT complex amplitude signal Ip. The OCT data acquisition components 440 may combine the Is and Ip OCT complex amplitude signals to generate the OCT amplitude signal I, as given by Equation 1 (described above with respect to FIG. 3).

The OCT data acquisition components 440 may then perform a logarithm scale calculation on the OCT signal I to generate A scan intensity profiles for the images. In certain embodiments, the OCT data acquisition components 440 may calibrate the interference signals to the wavenumber domain before performing dispersion compensation and other processing. The OCT A scan intensity profiles may be remapped to a logarithmic scale for display.

The control signals from the OCT light source 410 may be conveyed via the electrical cable 442. The overall operation of the SD-OCT subsystem 400 is controlled by controller 170, which sends control signals to the OCT data acquisition components 440 over electrical wires or cables.

In certain embodiments, the OCT data acquisition components digitize the raw fringes only, and then the fringes are sent to the controller 170 for signal processing, such as calibration of the interference signals to the wavenumber domain, dispersion compensation, apodization windowing, FFTs, OCT amplitude signal I calculation from Is and Ip, remapping I to a logarithmic scale, etc.

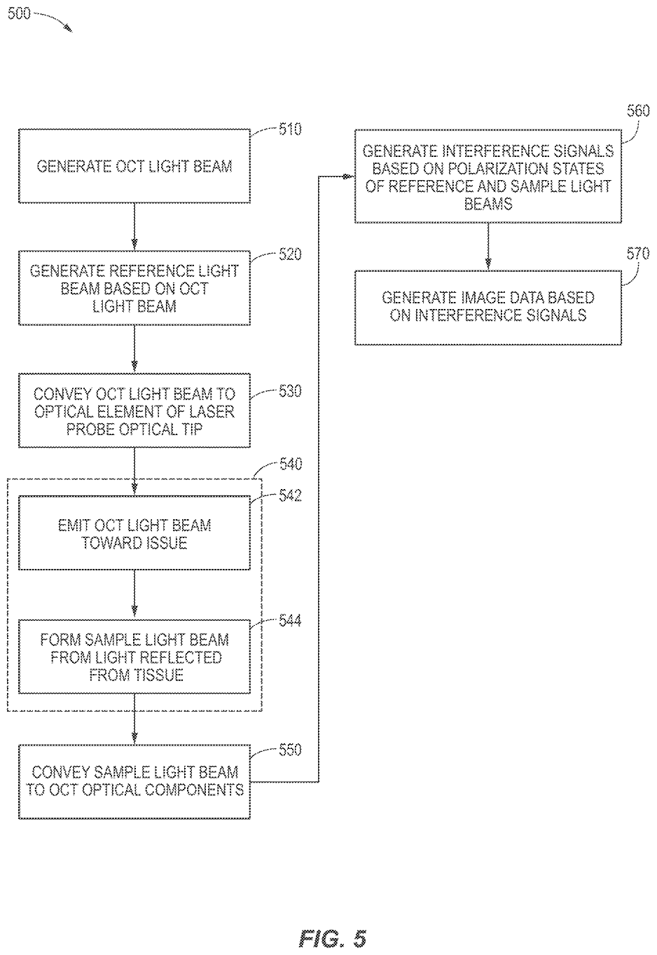

FIG. 5 depicts a flow diagram 500 describing certain functionality of the surgical laser system 100, 102 in accordance with certain embodiments of the present disclosure.

At block 510, an OCT light beam is generated by an OCT light source 152, as described above with respect to FIGS. 1A, 1B. In certain embodiments, the OCT light source 152 is OCT light source 310, such as a swept-source laser described above with respect to FIG. 3. In other embodiments, the OCT light source 152 is OCT light source 410, such as a broadband light source described above with respect to FIG. 4.

At block 520, a reference light beam is generated based on the OCT light beam. The reference light beam has elliptical polarization that includes two orthogonal polarization states.

At block 530, the OCT light beam is conveyed to an optical element 257 of the optical tip 240 of the laser probe 200 by an optical fiber 256, as described above with respect to FIG. 2.

Block 540 is performed by the optical element 257, and includes blocks 542, and 544.

At block 542, a portion of the OCT light beam is emitted toward tissue 20, as described above with respect to FIG. 2.

At block 544, a sample light beam is formed from light reflected from the tissue 20, as described above with respect to FIG. 2. The sample light beam has elliptical polarization that includes two orthogonal polarization states. There may have been retardation between these two orthogonal polarization states.

At block 550, the sample light beam is conveyed, by the optical fiber 256, from the optical element 257 to OCT optical components 154. In certain embodiments, the OCT optical components 154 are the OCT optical components 330 described above with respect to FIG. 3. In other embodiments, the OCT optical components 154 are the OCT optical components 430 described above with respect to FIG. 4.

At block 560, interference signals are generated, by the OCT optical components 154, based on the polarization states of the reference light beam and the polarization states of the sample light beam. In certain embodiments, the OCT optical components 154 are the OCT optical components 330 described above with respect to FIG. 3. In other embodiments, the OCT optical components 154 are the OCT optical components 430 described above with respect to FIG. 4.

At block 570, image data are generated, by OCT data acquisition components 156, based on the interference signals. In certain embodiments, the OCT data acquisition components 156 are the OCT data acquisition components 340 described above with respect to FIG. 3. In other embodiments, the OCT data acquisition components 156 are the OCT data acquisition components 440 described above with respect to FIG. 4.

### EXAMPLE EMBODIMENTS

In certain embodiments, a surgical laser system comprises a laser probe and an OCT subsystem.

The laser probe comprises an optical fiber configured to convey an optical coherence tomography (OCT) light beam, and a sample light beam having elliptical polarization comprising orthogonal polarization states, and an optical tip. The optical tip comprises a distal portion of the optical fiber, and at least one optical element configured to emit the OCT light beam toward tissue and receive reflected light from the tissue to form the sample light beam.

The OCT subsystem comprises an OCT light source configured to generate the OCT light beam, OCT optical components coupled to the OCT light source, and OCT data acquisition components coupled to the OCT optical components. The OCT optical components are configured to generate a reference light beam based on the OCT light beam, the reference light beam having elliptical polarization comprising orthogonal polarization states, transmit the OCT light beam from the OCT light source to the optical fiber in the laser probe, receive the sample light beam from the optical fiber in the laser probe, and generate interference signals based on the polarization states of the reference light beam and the polarization states of the sample light beam. The OCT data acquisition components are configured to generate image data based on the interference signals. For example, the OCT data acquisition components may be configured to digitize interference signals and generate image data based on the interference signals, or to digitize interference signals only and then transfer them to the display for signal processing.

In certain embodiments, a method for a surgical laser system comprises generating, by an OCT light source, an OCT light beam; generating a reference light beam based on the OCT light beam, the reference light beam having elliptical polarization comprising orthogonal polarization states; conveying, by an optical fiber, the OCT light beam to an optical element of a laser probe optical tip; emitting, by the optical element, the OCT light beam toward tissue, forming, by the optical element, a sample light beam from light reflected from the tissue, the sample light beam having elliptical polarization comprising orthogonal polarization states; conveying, by the optical fiber, the sample light beam from the optical element to OCT optical components; generating, by the OCT optical components, interference signals based on the polarization states of the reference light beam and the polarization states of the sample light beam; and generating, by OCT data acquisition components, image data based on the interference signals.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A surgical laser system, comprising:
a laser probe, comprising:
an optical fiber configured to convey an optical coherence tomography (OCT) light beam, and a sample light beam having elliptical polarization comprising elliptical polarization states, and
an optical tip comprising:
a distal portion of the optical fiber, and
at least one optical element configured to emit the OCT light beam toward tissue, and receive reflected light from the tissue to form the sample light beam; and
an OCT subsystem coupled to the laser probe, the OCT subsystem comprising:
an OCT light source configured to generate the OCT light beam,
OCT optical components coupled to the OCT light source, the OCT optical components configured to:
generate a reference light beam based on the OCT light beam, the reference light beam having elliptical polarization comprising elliptical polarization states,
transmit the OCT light beam from the OCT light source to the optical fiber in the laser probe,
receive the sample light beam from the optical fiber in the laser probe, and
generate interference signals based on the elliptical polarization states of the reference light beam and the elliptical polarization states of the sample light beam, and
OCT data acquisition components coupled to the OCT optical components, the OCT data acquisition components configured to generate image data based on the interference signals.

2. The surgical laser system of claim 1, wherein the elliptical polarization states of the reference light beam comprise:
a horizontal polarization state and a vertical polarization state, or
a polarization state P and a polarization state S and the elliptical polarization states of the sample light beam comprise a horizontal polarization state and a vertical polarization state, or
a polarization state P and a polarization state S.

3. The surgical laser system of claim 1, wherein the optical element comprises a polished angled tip that is formed in the distal portion of the optical fiber.

4. The surgical laser system of claim 1, wherein the optical element comprises a focusing lens that is attached to an end of the distal portion of the optical fiber using an optical adhesive that has a refractive index that matches a refractive index of the optical fiber.

5. The surgical laser system of claim 1, wherein the optical element comprises an articulated prism that is attached to an end of the distal portion of the optical fiber using an optical adhesive that matches a refractive index of the optical fiber.

6. The surgical laser system of claim 1, wherein:
the OCT optical components comprise:
a first variable optical attenuator (VOA) coupled to the OCT light source,
a first 2×2 fiber coupler coupled to the first VOA and the optical fiber in the laser probe,
a second VOA coupled to the first 2×2 fiber coupler,
a reference delay line (RDL) coupled to the second VOA, and
a second fiber coupler coupled to the first 2×2 fiber coupler and the RDL;
the first 2×2 fiber coupler is configured to:
transmit the OCT light beam from the OCT light source to the optical fiber in the laser probe,
transmit the OCT light beam to the second VOA,
receive the sample light beam from the optical fiber in the laser probe, and
transmit the sample light beam to the second 2×2 fiber coupler; and
the reference light beam is generated by the second VOA and the RDL.

7. The surgical laser system of claim 6, wherein:
the OCT light source is a swept-source laser; and
the second fiber coupler is 2×2 fiber coupler configured to:
receive the reference light beam from the RDL and the sample light beam from the first 2×2 fiber coupler,
combine the reference light beam and the sample light beam,
divide the combined light beams into a first light beam and a second light beam, and
output the first light beam and the second light beam.

8. The surgical laser system of claim 7, wherein the OCT optical components further comprise:
a first polarization beam splitter (PBS) coupled to the second 2×2 fiber coupler, the first PBS configured to receive the first light beam from the second 2×2 fiber coupler, divide the first light beam into an S₁ polarization channel light beam and a P₁ polarization channel light beam, and output the S₁ polarization channel light beam and the P₁ polarization channel light beam;
a second PBS coupled to the second 2×2 fiber coupler, the second PBS configured to receive the second light beam from the second 2×2 fiber coupler, divide the second light beam into an S₂ polarization channel light beam and a P₂ polarization channel light beam, and output the S₁ polarization channel light beam and the P₁ polarization channel light beam;
a first balanced detector coupled to the first PBS and the second PBS, the first balanced detector configured to receive the S₁ polarization channel light beam and the S₂ polarization channel light beam, generate an S polarization channel interference signal, and provide the S polarization channel interference signal to the OCT data acquisition components; and
a second balanced detector coupled to the first PBS and the second PBS, the second balanced detector configured to receive the P₁ polarization channel light beam and the P₂ polarization channel light beam, generate a P polarization channel interference signal, and provide the P polarization channel interference signal to the OCT data acquisition components.

9. The surgical laser system of claim 6, wherein:
the OCT light source is a broadband light source; and
the second fiber coupler is 2×1 fiber coupler configured to:
receive the reference light beam from the RDL and the sample light beam from the first 2×2 fiber coupler,
combine the reference light beam and the sample light beam into a light beam, and
output the light beam.

10. The surgical laser system of claim 9, wherein:
the OCT optical components further comprise:
a PBS coupled to the second 2×2 fiber coupler, the PBS configured to receive the first light beam from the second 2×2 fiber coupler, divide the first light beam into an S polarization channel light beam and a P polarization channel light beam, and output the S polarization channel light beam and the P polarization channel light beam,
a collimator comprising a first collimator and a second collimator coupled to the PBS, the first collimator configured to receive the S polarization channel light beam, and generate a free space S polarization channel collimated light beam, the second collimator configured to receive the P polarization channel light beam, and generate a free space P polarization channel collimated light beam,
a transmission grating configured to receive and diffract the S and P polarization channel collimated light beams into multiple light beams based on wavelength, and
an imaging lens comprising one or more lenses that focuses the multiple light beams generated by the transmission grating onto an image capture device;
the first and second collimators are tilted with respect to each other to separate the S and P polarization channel light beams on the image capture device after going through the transmission grating and imaging lens; and
the image capture device is configured to generate interference signals, and provide the interference signals to the OCT data acquisition components.

11. The surgical laser system of claim 1, wherein:
the image data is an A scan providing a depth profile of the tissue at a particular location; or
the image data is a B scan comprising a plurality of A scans providing a cross-sectional view of the tissue along a lateral direction.

12. The surgical laser system of claim 1, further comprising:
a treatment light source configured to generate a treatment light beam; and
an illumination light source configured to generate an illumination light beam,
wherein the laser probe further comprises:
a treatment optical fiber coupled to the treatment light source,
an illumination optical fiber coupled to the illumination light source,
wherein the optical tip of the laser probe further comprises:
a distal portion of the treatment optical fiber configured to emit the treatment light beam toward the tissue, and
a distal portion of the illumination optical fiber configured to emit the illumination light beam toward the tissue.

13. The surgical laser system of claim 12, further comprising:
a camera interface configured to process video signals,
wherein the laser probe further comprises:
a camera located in the optical tip, the camera configured to generate the video signals, and
an electrical cable coupled to the camera and the camera interface.

14. The surgical laser system of claim 12, further comprising:
a camera interface configured to process video signals, the camera interface comprising a camera configured to generate the video signals,
wherein the laser probe further comprises an optical fiber bundle coupled to the camera interface.

15. A method for a surgical laser system, comprising:
generating, by an OCT light source, an OCT light beam;
generating a reference light beam based on the OCT light beam, the reference light beam having elliptical polarization comprising elliptical polarization states;
conveying, by an optical fiber, the OCT light beam to an optical element of a laser probe optical tip;
emitting, by the optical element, the OCT light beam toward tissue;
forming, by the optical element, a sample light beam from light reflected from the tissue, the sample light beam having elliptical polarization comprising elliptical polarization states;
conveying, by the optical fiber, the sample light beam from the optical element to OCT optical components;
generating, by the OCT optical components, interference signals based on the elliptical polarization states of the reference light beam and the elliptical polarization states of the sample light beam; and
generating, by OCT data acquisition components, image data based on the interference signals.

16. The method of claim 15, wherein the elliptical polarization states of the reference light beam comprise:
a horizontal polarization state and a vertical polarization state, or
a polarization state P and a polarization state S and the elliptical polarization states of the sample light beam comprise a horizontal polarization state and a vertical polarization state, or
a polarization state P and a polarization state S.

17. The method of claim 15, wherein the optical element comprises a polished angled tip that is formed in a distal portion of the optical fiber.

18. The method of claim 15, wherein the optical element comprises a focusing lens that is attached to an end of a distal portion of the optical fiber using an optical adhesive that has a refractive index that matches a refractive index of the optical fiber.

19. The method of claim 15, wherein the optical element comprises an articulated prism that is attached to an end of a distal portion of the optical fiber using an optical adhesive that matches a refractive index of the optical fiber.

20. The method of claim 15, wherein the OCT light source is a swept-source laser or a broadband light source.
